(19) United States
(12) Patent Application Publication
(10) Pub. No.: US 2012/0128827 A1
(43) Pub. Date: May 24, 2012

(54) FUNCTIONAL FEEDS WITH HIGH SOY FLOUR, STARCH AND Bacillus SPP CONTENT

(75) Inventor: JOSÉ LEONEL OCHOA, ENSENADA (MX)

(73) Assignee: CENTRO DE INNOVACION BIOTECHOLOGIVA, A.C., ENSENADA (MX)

(21) Appl. No.: 13/300,271

(22) Filed: Nov. 18, 2011

(30) Foreign Application Priority Data
Nov. 19, 2010 (MX) MX/A/2010/013458

(51) Int. Cl. A23L 1/30; C12N 1/21; C12N 1/20

(52) U.S. Cl. 426/61; 435/252.5; 435/252.31

(57) ABSTRACT

The present product (“invention”) corresponds to a new strain of Bacillus subtilis NRRLB-50213 which produces secondary metabolites, bioactive substances and biodegrading enzymes. This strain of bacteria and/or its derivatives has/have the capacity to form functional feeds with high starch and/or soy flour content, as well as inhibiting the growth of pathogenic microorganisms.

Functional feeds formulated with the NRRLB-50213 strain and high concentrations of vegetable flours have the capacity to generate growth, weight gain and survival equal to or greater than those generated by feeds which contain high fish flour or other animal protein content.

Included in this invention are methods and formulas which increase growth, weight gain and survival percentage, since the decomposition and assimilation of formulated functional nutrients based on vegetable flour and the NRRLB-50213 strain of Bacillus subtilis are improved and maximized. In addition to this, the functional feeds employed in this product protect animals, since they prevent the occurrence of diseases caused by pathogenic microorganisms

### TECHNICAL FIELD OF THE INVENTION

The innovative product we are going to discuss here is related to the technical field of nutrition, primarily that field which develops feeds with a high soy flour or starch content, as well as bacterial species of the genus Bacillus, especially B. subtilis.

These feeds should match or exceed growth, weight gain and survival percentage in animals that consume them, compared with animals fed with high contents of animal protein.

### BACKGROUND

Animal nutrition has a preponderant place in the biological processes of organisms; it allows them to satisfy their energy requirements, making it possible to carry out their vital functions.

A Functional Food (FF) has the characteristic of generating benefits beyond those of conventional feeds. Their components have to be economical and easy to obtain. FF's must be harmless to animals and human beings. This functionality must be reflected in an improvement in the percentage of survival, greater growth and weight gain among the animals which consume it. This means that carbohydrates, proteins and lipids are well-assimilated and channeled to the corresponding primary functions.

Fish Flour (FM)

Currently, animal feeding is the largest expense at a production farm, requiring investments that range from 50% to 70% of the total. This is mainly due to the use of very costly, but easily-assimilable substrata such as fish flour, which have a high protein content (Bautista-Teruel et al., 2003; Kureshy and Davis, 2002; Pascual et al., 2004b).

Aquaculture is a rapidly-growing activity and is also the greatest consumer of fish flour in the world. But it is not the only one (FAO, 2006; Ochoa and Olmos, 2006). Diets for shrimp and carnivorous fish contain from 40% to 60% of fish flour (Hardy and Barrows, 2002; Pascual et al., 2004; Wilson, 2002; Peres and Oliva, 2002). In addition, stockbreeding, pig breeding and the pet food industry, among other things, use—or used to use—large percentages of fish flour in their formulas.

The high demand and limited production of fish flour have caused a disproportionate increase in the price of this ingredient in recent years, causing prices to rise to such as an extent as to almost make the use of fish flour in animal food prohibitive, which has caused a serious economic problem for producers in the areas of aquaculture, fishing and pet food (Tacon and Metian, 2008).

In addition, the limited capacity of carnivorous fish (<12%), shrimp (<20%) and, in general, of animals (<20%), to digest starch in their diet due to the lack of appropriate alpha-amylases (alpha 1-4 and/or alpha 1-6), cause fish flour to be used by animals as a source of protein and energy, making feeding less efficient and more costly (Arellano and Olmos, 2002; Enes et al., 2008; German et al., 2004: Hemre et al., 1996; Le Chevalier and Van Wormhoudt, 1998; Pascual et al., 2004a; Peres and Oliva, 2002; Rosas et al., 2000).

For this reason, affordable alternative ingredients are needed to reduce the costs related to the preparation of animal feeds; which contain high levels of proteins and carbohydrates; these need to also (in addition to being inexpensive) be easily assimilable by animals.

Soy flour and starch (corn or wheat flour) are the ingredients currently used to replace fish flour as a source of protein and energy, respectively; however, these are difficult for animals to digest and assimilate and contain anti-nutritional compounds which cause serious health problems, for which reason its usage is limited to low concentrations in diets (Amaya et al., 1997a; Arockia et al., 2007: Desrsjant-Li, 2002; Foster et al., 2002; Krogdahl et al., 2005; Ochoa and Olmos, 2006).

In this context, to date there has been no practical, comprehensive solution to producing economically-competitive, highly-assimilable food which is innocuous for animals and profitable for the producer.

The unique aspect of the innovative product we have produced is that it makes it possible to formulate feeds with high soy flour and starch contents and a strand of Bacillus subtilis, which feeds can be digested and assimilated by animals, causing positive effects on growth, weight-gain and survival, without adverse health reactions, and which is profitable for the producer—which has not been possible to date.

Soy Flour

Soy flour contains high concentrations of protein (˜40%) and carbohydrates (˜16%). However, it is well known that soy flour also contains several antinutritional compounds, which produce adverse effects in animals—for which reason the use of soy flour is limited to concentrations no greater than 20% in feeds (Dersjant-Li, 2002; Heikkinen et al., 2006).

Among these antinutritional compounds are a trypsine inhibitor, allergenic proteins and oligosaccharides (Cordel, 2004; Dunsford et al., 1989), which can only be eliminated by means of long, costly processes (Dersjant-Li, 2002; Nora et al., 2006; Zhu et al., 2008). Analysis of fermented soy has demonstrated the elimination of most antinutritional compounds through certain species of bacteria which are capable of eliminating these compounds (Nora et al., 2006; Zhu et al., 2008). However, the use of fermented soy flour is not cost-effective and is impractical at a commercial level of food production.

Another alternative is to use Soy Protein Concentrate, which has high concentrations of highly-assimilable protein and has been cleansed of antinutritional compounds by means of chemical processes. However, its use in commercial feeds is even more costly than fish flour itself (Dersjant-Li, 2002).

Starch (Corn and Wheat Flour, Among Others)

One of the main ingredients in food(s), but not much utilized by animals, is starch (Arockia et al., 2007; German et al., 2004). Starch is comprised of linear (amylose) and branched (amilopectine) structures, which have alpha 1-4 and alpha 1-6 links, respectively. Alpha 1-4 links are difficult for monogastric animals to break. Among these are carnivorous fish, shrimp and pigs. In addition, alpha 1-6 links are impossible for monogastric animals to break, and their 2002; Le Chevalier and Van Wormhoudt, 1998; Pascual et al., 2004a; Peres and Oliva, 2002; Rosas et al., 2000).

Starch is limited to concentrations lower than 12% in formulas for carnivorous fish, since it causes adverse reactions in regard to the assimilation of other ingredients and to animals' health, limiting their growth and weight gain (Arockia et al., 2007; Enes et al., 2008; German et al., 2004; Hemre et al., 2002; Peres and Oliva, 2002).

In addition, it has been shown that the starch content in shrimp food should not be greater than 20% (Rosas, et al., 2000). This is due mainly to the lack of the alpha amylases needed by cultivated shrimp to digest this ingredient (Arellano and Olmos, 2002; Le Chevalier, 1998; Pascual et al., 2004).

The toxic effects of starch have not been studied much; however, poor assimilation of starch causes the animal to use protein as an energy source, limiting its use to growth and immunity purposes (Pascual et al., 2004). For this reason, starch concentrations greater than 20% are not adequately assimilated by the animal; amounts in excess of this are an unnecessary expense in the preparation of the formula and can cause adverse effects on the health of animals that eat it (Ochoa and Olmos, 2006; Pascual et al., 2004).

Most commercial diets for shrimp contain from 30% to 40% of starch, mainly due to the fact that this ingredient is the cheapest and easiest to get. However, starch is mainly used as a filler component in food formulas' and to provide agglutination of the food that is formulated. However, the energy content present in starch is very high and, if it could be assimilated, would have a great impact on the growth and health of the animal; it would make it possible to avoid unnecessary expenses in formulating the food and in nutrition in general (Ochoa and Olmos, 2006; Pascual et al., 2004a).

As is the case with fermented soy flour, there has been research in which starch has been fermented using Lactobacillus, improving its digestibility—according to tests performed in vitro (U.S. Patent number 2003/0059416A1). However, as is the case with fermented soy, the use of fermented starch is not cost-effective and is impractical at the time of formulating and producing food at a commercial level. In addition, this research has shown no evidence of fermented starch producing benefits in terms of the growth or health of cultivated animals.

Our invention produced beneficial results in terms of growth, weight gain and percentage of survival in the animals experimented upon—pigs, carnivorous fish and cultivated shrimp, who consumed formulas with high concentrations of soy flour, starch and a strain of Bacillus subtilis. Results like this cannot be found in the literature on this subject, nor in any patent document extant at this time.

The Use of Bacillus

Soy flour and starch fermentation processes using species of Bacillus y Lactobacillus, respectively, have shown that these species can be used separately to improve the digestibility of these ingredients. However, in the research just mentioned, only in vitro digestibility experiments were performed; in vitro experiments did not show benefits in terms of the growth, weight gain and survival that animals can obtain.

Bacillus subtilis is a gram-positive bacteria which is generally recognized to be safe by the FDA (Sonnenschein et al., 1993). This bacteria has the capacity to produce and secrete a large amount of enzymes (proteases, carbohydrases, llipases, among others). They can also produce antimicrobial peptides and stimulate animals' immune systems (Arellano and Olmos, 2002; Ochoa and Olmos, 2006; Sonnenschein et al., 1993).

As has been mentioned, various research has shown the ability of B. subtilis and Lactobacillus to digest soy flour and starch respectively, by means of fermentation processes. In regard to fermented soy flour, elimination of antinutritional compounds and improvement in digestibility of starch in vitro has been demonstrated, due to the presence of enzymes in both processes. However, both fermented soy flour and fermented starch have not been used together and/or separately to produce formula feeds at a commercial level; this is because it is impractical and cost-ineffective at the time of preparation.

To date, no fermentation process has mixed both ingredients; therefore there is no evidence of a bacterial strain or mixture of strains improving the digestibility of soy flour and starch at the same time.

Due to how impractical and cost-ineffective the fermentation process prior to formulating the food is, unfermented soy flour and starch are still being used in commercial feeds, and the same are still causing the same negative effects on growth, weight gain and survival among the animals—when these ingredients are used in large concentrations.

In our invention, the B. subtilis NRRL B-50213 strain made it possible to formulate animal feeds with high soy flour and starch contents, with which the growth, weight gain and survival percentages of animals were improved considerably.

The formulas of our invention which included the NRRL B-50213 strain of B. subtilis produced better results than those generated with feeds formulated with fish and vegetable flours, but without the inclusion of B. subtilis NRRL B-50213. In addition, the food conversion factor and health status were improved with our formulas, which was demonstrated in in vivo experiments. Another novel aspect of our invention is the ability to formulate highly-assimilable, healthy and economically-viable feeds for animal use, without the need for prior fermenting processes.

Purpose of the Invention

The present invention had as its purpose the provision of a strain of Bacillus subtilis and/or its derivatives, which has the capacity to produce enzymes such as carbohydrates, proteases and lipases, which maximize the breakdown and assimilation of elements. This strain of B. subtilis also has the capacity to inhibit the growth of pathogenic microorganisms and stimulate the animal's immunological system; it thereby prevents the occurrence of diseases and, as a result, it improves survival percentages. This strain was deposited as number NRRL B-50213. The invention also includes the strain's derivatives—such as its mutations, its supernatant and its culture.

The present invention had as purpose the provision of a strain of Bacillus subtilis, which maximizes degradation and assimilation of food formulated with a high content of soy flour and/or starch. That is reflected in growth, weight gain and percentage of survival equal or higher than the those obtained with fishmeal and higher than the those obtained with vegetal flours without the NRRL B-50213 B. subtilis strain. Furthermore, it does not create adverse effects for the health of the evaluated animals such as pig, carnivorous fish and shrimp.

Another purpose for this invention is that of providing a mixture which contains the NRRL B-50213 strain and/or its derivatives, as well as a high soy flour (>20%) and/or starch (>12%) content, in which the proteins, carbohydrates, enzymes and bioactive contents bring about growth, weight gain and survival percentages equal to or greater than those obtained with fish flour, and greater than those obtained with vegetable flours without the NRRL B-50213 strain. Furthermore, it does not cause adverse effects on the health of the animals evaluated: pigs, carnivorous fish and shrimp (sic).

Another purpose for this invention is that of providing a mixture which contains the NRRL B-50213 strain and/or its derivatives, as well as a high soy flour (>20%) and/or starch (>12%) content, where this formulation has the capacity to inhibit the growth of pathogenic microorganisms and stimulate the immunological system of the animal, by which means it might prevent the occurrence of illnesses in animals and increase the percentage of survival—mainly that of pigs, carnivorous fish and shrimp.

The invention also involves a method of preparing the food consisting on applying an effective amount of the NRRL B-50213 strain and/or its derivatives to the other ingredients, which are: soy flour, cotton and other minor compounds, which gives rise to a mixture (formulation). When this mix is consumed by the animals; it generates growth, weight gain and survival percentages equal or higher than those obtained with fish flour and higher than the those obtained with vegetal flours without the strain. Furthermore, it does not create adverse effects for the health of the evaluated animals such as pig, carnivorous fish and shrimp.

Therefore, the invention also involves a method of preparing the food which consists of applying an effective amount of the NRRL B-50213 strain and/or its derivatives to the other ingredients, which are: soy flour, cotton and other minor compounds, which gives rise to a mixture (formulation). This formula has the capacity to inhibit the growth of pathogenic microorganisms and stimulate the animal's immunological system, thereby preventing the occurrence of illnesses in animals, and it increases the percentage of survival—mainly of pigs, carnivorous fish and shrimp.

### DESCRIPTION OF THE INVENTION

The characteristic details of the present invention are clearly shown in the following description and in the accompanying figures and tables.

Bacillus subtilis is the best-characterized member of the Gram-positive bacteria. Its genome consists of 4,214,810 base pairs (Kunst et al., 1997). It is aerobic, in the shape of a cane, and in adverse conditions it sporulates. It is commonly found in soil and water, and in association with plants and animals (Arellano and Olmos, 2002; Garcia and Olmos, 2007). It is an important source of industrial enzymes such as amylases, proteases and lipases (Ochoa and Olmos, 2006). It produces a large quantity of natural antimicrobes against fungi and bacteria (Sonnenschein et al. 1993).

B. subtilis has been used as a host in the production of heterologous proteins (Olmos and Contreras, 2003). B. subtilis has attracted a lot of attention due to its safety and innocuousness and is considered a “Generally Recognized as Safe (GRAS)” organism, for plants, animals and humans, by the Food and Drug Administration (FDA) (Westers, et. al, 2004).

Isolation and Phenotypical Identification of the B. Subtilis NRRL B-50213 Strain.

For the isolation and selection of strains of B. subtilis of importance for the field of nutrition, 50 soil samples, collected in the region of Baja California, Mexico, were used. The samples collected were treated by the spore-selection method and cultivated in Petri boxes with Luria Bertani (LB) for 24 hours at 37° C. (Sonnenschein et al., 1993). The colonies were phenotypically sorted and their enzymatic activities were measured in agar plates which were heated to 37° (“incubadas a 37° C.) for 24 hours. From the colonies which were cultivated, samples were taken of each that had different phenotypical and enzymatic characteristics (proteases, lipases and carbohydrases) and these were tinted with Gram's tincture

Molecular Identification of the NRRL B-50213 Strain

Extraction and Purification of DNA

The isolated strains were cultivated in flasks with LB media at 300 rpm, at 37° C. for 12 hours. Chromosomal DNA was extracted and purified using the phenol-choroformal and ethanol method (Sambrook et. al, 1989).

Amplification by PCR and Sequencing

The 16S rDNA gene was amplified by the PCR technique, using the following reaction conditions: genomic DNA, first universal 16Sf and a first specific for Bacillus (Arellano and Olmos, 2002). The PCR products of selected strains B, C and H were purified in our laboratory and sequenced at the San Diego State University Microchemical Core Facility. Finally, the sequences were compared with known sequences and registered at the Gene Bank of the National Center for Biotechnology Information (NCBI) (http://www.ncbi.nlm.nig.gov/). Strain B was identified as B. subtilis containing a likeness equal to 98%. Strains C and H were identified as B. megaterium, with a 95% likeness. The sequenced bacteria were preserved at −70° C. in 2 ml cryovials.

Aiding in the Digestibility of Carbohydrates.

The genus Bacillus secrets a significant amount of enzymes, among which are carbohydrases, proteases and lipases, which can aid in the digestion of carbohydrates, proteins and lipids, respectively (Ochoa and Olmos, 2006). In the case of carbohydrates, B. subtilis strain NRRL B-50213 and the carbohydrates it produces, improved the digestibility of the starch that comes from different vegetable flours (Table 1).

Maximizing the breakdown and assimilation of starch represents a healthy solution, of economical importance, in animal feeding, since it would eliminate the toxic effects of starch and at the same time diminish the amount of fish flour and/or animal protein added to commercial formulas (Ochoa and Olmos, 2006).

In Table 1 the results in terms of starch breakdown are presented in regard to the selected strains, using different vegetable flours. In this table it can be observed that strains B, C and H present the largest breakdown area, which indicates their great capacity to digest starch. Considering that the flour most commonly used in animal food is wheat flour, strain B was selected for its subsequent cultivation and testing.

TABLE 1
Capacities of isolated strains to use sources of Starch.

| Strain | Cellulose Flour | Corn Flour | Wheat Flour |
|---|---|---|---|
| A | + | + | + |
| B | ++ | +++ | +++ |
| NRRLB-50213 | | | |
| C | ++ | ++ | + |
| D | + | ++ | + |
| E | + | ++ | + |
| F | ++ | + | + |
| G | + | ++ | + |
| H | ++ | ++ | + |

Degradation area in centimeters (1.5-1.0) +++ (0.9-0.5) ++ (0.4-0.1) +

In order to find out the type of carbohydrolases present in the strains, specific substrates were used to identify alpha glucocidase and alpha galactocidase enzymes. Table 2 shows that the B (NRRLB-50213) strain subject to this patent produces alpha 1-4 and alpha 1-5 glucocidase enzymes. Furthermore, this strain also has the capability to flow with melobiose and rafinose with alphagalactoside links. These results demonstrate that the NRRL B-50213 strain can break down starch and carbohydrates considered as anti-nutritional that can be found in soy flour.

CHART 2
Alphaglucosidase and alphagalactoside
production in isolated strains.

| | Amilose | | Melibiose | |
|---|---|---|---|---|
| | and x- | Amilopectine | and X- | Raffinose y |
| Strain | alpha-glu | and X-alpha-glu | alpha-gal | X-alpha-gal |
| A | + | + | + | + |
| B | ++ | ++ | + | ++ |
| NRRLB-50213 | | | | |
| C | − | − | − | − |
| D | − | − | − | − |
| E | + | + | − | + |
| F | + | + | − | − |
| G | − | − | + | + |
| H | + | + | + | + |

Coloration area in centimeters (1.5-1.0) +++ (0.9-0.5) ++ (0.4-0.1) +

The aforementioned suggests that by adding the NRRL B-50213 strain to food, better breakdown of the carbohydrates found in vegetable flours can be achieved and, as a result, more energy will be available to the animal. This means a decrease in feeding costs and higher growth. This is due to the reduction of the protein consumption to obtain energy (Pascual et al., 2004a). This way, the concentrations of inexpensive energy sources such as starch may be increased; this is because, in principle, there would be no toxic effects for the animal (Ochoa y Olmos, 2006).

Auxiliary in Protein Digestion

The capability of the NRRL B-50213 strain to produce enzymes such as protease, carbohydrolases and lipases allows the strain to be utilized for several purposes in the same food. The quantity and quality of the protein in a diet is a key factor for animal growth. Before, in animal farming, the protein used primarily came from fish meal; however, the scarcity and high cost of fish meal have limited its usage greatly.

Soy flour as a protein source is restricted due to the anti-nutrients and allergic proteins contained in it. This makes the digestion of soy flour harder and it is a cause of health problems in animals. For this reason soy flour is to be or should be added in concentrations not greater than 20% in food. In this way, the results in Chart 3 show that the NRRL B-50213 strain has the capability of degrading high concentrations of soy and fishmeal. Therefore, this strain may enhance the digestive system of the animal by helping the animal to digest and, in consequence, to assimilate proteins regardless of where they came from. This would generate higher animal growth and fattening by using the same amount of animal or vegetable protein, added to the food.

CHART 3
Protease and lipase activity in selected strains

| Strain | Soy flour | Fishmeal | Fish oil | Corn oil |
|---|---|---|---|---|
| A | + | + | + | + |
| B | +++ | +++ | ++ | ++ |
| NRRL B-50213 | | | | |
| C | ++ | ++ | + | + |
| D | ++ | + | + | ++ |
| E | ++ | + | + | + |
| F | ++ | + | + | + |
| G | + | + | + | + |
| H | ++ | ++ | + | + |

Growth area in centimeters (2.5-1.5) +++ (1.5-0.5) ++ (0.5-0.1) +

Supports the Digestion of Lipids

Besides supporting the degradation of carbohydrates and proteins, the NRRL B-50213 strain that is the subject of this invention showed an excellent capability of assisting in the degradation of lipids of both animal and vegetable origin (Chart 3). The above could mean that the inclusion of the NRRL B-60213 strain in feeds could increase the digestion and assimilation capability of the lipids consumed by the animals. This would generate a greater energy amount available with the same amount of lipids, regardless of the source and, as a consequence, a decrease in feeding costs.

Because of the results shown above, the B strain was deposited in the Agricultural Research Service (ARS) Patent Culture Collection of the United States of America on the 20^{th} day of January of 2009, under the International Treaty of Budapest for patent purposes. This strain received access number NRRL-B0213, whose features are the following: it was classified as Bacillus subtilis by sequentiation of its 16S (DNA, is Gram (+)), it shows a bacillary form and has a lateral endospore. The colonial morphology of the strain is furrowed, has irregular edges and it an opaque white color.

The strain, during its exponential growth, shows a chain growth, however during the stationary phase it shows an individual bacillary form.

Feeding Formula

For this invention, high concentrations of vegetable flour were used as a source of carbohydrate and protein with higher levels than those digestible and assimilable by animals. It is important to mention that when the NRRL B 50213 strain was supplemented with several formulations, positive effects were reflected as improvement in growth, weight gain and survival percentage. For this reason, the animals fed with higher concentrations of vegetable flour and sufficient amount of the mix or the new strain of B. subtilis NRRL B 50213, were those that showed better performance.

The procedure followed for the formulation was to mix the ingredients and then add the bacterial strains, or if using a commercial formulation, by adding the NRRL B50213 strain only. The feeding formulas were dried at 70° C. for 24 hours, until humidity was 10%.

### EXAMPLE 1

Degradation and Assimilation of High Concentrations of Starch in Carnivorous Fish

There are reports that carnivorous fish are not able to digest starch in concentrations higher than 12% for this generates serious health problems in these animals. The carnivorous fish that were used were farmed as follows: 300 hundred fish were selected at random and were distributed in pools. The fish were fed with a basic diet containing varying concentrations of starch and the NRRL B-50213 strain (Chart 4). Fish were fed 3 times per day until apparent satiety and the experiment lasted 8 weeks.

CHART 4
Approximate composition of diets used for farmed White Sea Bass

APPROXIMATE

| COMPOSITION | Diets | | | | |
|---|---|---|---|---|---|
| g/100 g | DC | DP10 | DP14 | DP18 | DP22 |
| Protein (fishmeal) | 56.5 | 56.1 | 56.0 | 54.2 | 55.9 |
| Lipids (fish oil) | 19.3 | 18.3 | 15.0 | 15.0 | 14.3 |
| Ashes | 9.8 | 10.5 | 10.2 | 10.8 | 8.9 |
| Corn starch | 10.3 | 10.5 | 13.9 | 18.8 | 21.4 |
| Caloric Energy g⁻¹ | 5076 | 5102 | 5037 | 5071 | 5066 |
| NRRL B-50213 strain | 0.00 | 0.01 | 0.01 | 0.01 | 0.01 |

Table 5 shows the growth parameters of White Sea Bass (Atractoscion nobilis). The DP22 experimental diet of higher starch percentage had the best results for the fish parameters. Therefore, it is demonstrated that NRRL B-502213 strain, when added to starch diets at percentages greater than 12%, does increase the degradation and assimilation of this complex carbohydrate in carnivorous fish and results in significant benefits in all the evaluated parameters.

TABLE 5
Growth, weight gain and survival in cultures of Corvina blanca
with high concentrations of starch and the NRRL B-50213 strain.

| Parameters | DC | DP10 | DP14 | DP18 | DP22 |
|---|---|---|---|---|---|
| Initial Weight | 9.6 ⁺₋0.07 | 9.5 + 0.04 | 9.5 + 0.2 | 9.437 + 0.2 | 9.5 + 0.11 |
| Final Weight | 18.3 + 1.04 | 21.4 + 0.15 | 21.7 + 0.5 | 27.5 + 1.88 | 33.8 + 1.47 |
| Weight gained | 8.6 + 1.02 | 11.9 + 0.11 | 12.2 + 0.78 | 18.1 + 1.84 | 24.3 + 1.38 |
| Initial Light | 9.02 + 0.06 | 9.03 + 0.04 | 8.87 + 0.16 | 8.99 + 0.09 | 9.04 + 0.05 |
| Final Light | 11.7 + 0.05 | 11.9 + 0.02 | 12.1 + 0.10 | 12.9 + 0.22 | 13.7 + 0.13 |
| Light | 2.6 + 0.07 | 2.9 + 0.02 | 3.3 + 0.18 | 3.9 + 0.16 | 4.6 + 0.08 |
| Consumption | 14.0 + 0.72 | 14.7 + 0.05 | 16.4 + 1.18 | 18.1 + 2.34 | 20.7 + 1.01 |
| Survival rate | 90.0 + 10 | 95 + 10 | 95.0 + 5 | 95.0 + 5 | 100 + 0 |

These results show that the NRRL B 59213 strain considerably improved the degradation and assimilation of carbohydrates, protein and lipids contained in the diet; which was expected due the excellent results obtained in the experiments performed in-vitro (Charts 1, 2 y 3).

In this work, the more starch was added to the diet the better were the results obtained for the physiological and physical parameters; this means that with the formula and the NRRL B-50213 strain, the fish were able to digest and assimilate higher concentrations of starch with no problems. (Chart 5).

With our formulation, the animal is using starch as a source of energy instead of protein as generally occurs with conventional feeds. For this reason, the protein added to these feeds is exclusively intended for animal growth and not to supply energy requirements; because of this, growth and weight gain were very significant. This makes the strain and/or the mix, when used in an effective amount, a good alternative for the degradation and assimilation of starch in levels greater than 12%, which was impossible in the past. Besides improving the degradation of lipids and proteins of the diet, the strain improved the use of the nutrients by the animal.

Due to these results, adding fishmeal to carnivorous fish diets may be reduced and the use of carbohydrates such as starch and other complex polysaccharides may be increased, as they are contained in vegetable flours, provided that they are supplemented with the NRRL-B0213 strain which aids in the degradation and assimilation of food and boosts growth, weight gain and survival rate of the animal.

### EXAMPLE 2

Usage of High Concentrations of Soy Flour and Starch for Shrimp

In order to demonstrate that the NRRL-B-50213 strain has the same capability to maximize the degradation and assimilation of starch in shrimp for carnivorous fish, simulations where performed using shrimp of the most important culture. Chart 6 shows the approximate composition of the diets used. Commercial food was used as the control diet and a formulation of soy flour and starch created for this project, added to the NRRL B-50213 strain. It is worth mentioning that the level of starch of our formula reached 50% of the total weight, while the recommended concentration for shrimp food is 20% (Pascual et al, 2004a). Likewise, it is important to point out that in our formula, all protein used was from soy flour; for this reason fish flour was not used, as opposed to the commercial diet. We were able to prove that the composition of carbohydrates in the commercial diet surpasses the 20% recommended, but still it contained ten units less than our formula (Chart 6).

CHART 6
Proximal composition of diets used for white shrimp.

| | | | Basal Diet + NRRL |
|---|---|---|---|
| Ingredients | Basal Diet | Commercial Diet | B-50213 |
| Protein | 27.41 | 36.39 | 27.41 |
| Lipids | 6.46 | 3.98 | 6.46 |
| Carbohydrates | 49.50 | 38.42 | 49.50 |
| Humidity | 11.34 | 9.88 | 11.34 |
| Ashes | 5.29 | 11.33 | 5.29 |
| B. subtilis | 0.00 | 0.00 | 0.01 |

Food was consumed by shrimp 3 times a day, within a period of 2 months. Chart 7 shows how, even though all formulas produced an increase of weight, the formula containing NRRL B-50213 produced the best results. Additionally, the food conversion factor (FCA) was decreased with respect to commercial food and a significant effect was created in the survival percentage. The results shown below prove that the NRRL B-50213 strain is a practical and effective solution for the use of inexpensive vegetal flours as an energy and protein source for shrimp, decreasing production expenses caused by food.

CHART 7
Weight gain, survival and food conversion factor,
of farmed shrimp fed with diets shown in Chart 6.

| | | | Basal Diet + NRRL |
|---|---|---|---|
| Parameters | Basal (B) | Commercial | B-50213 |
| Initial | 5.98 +− 0.22^{a} | 6.06 +− 0.18^{a} | 5.96 +− 0.20^{a} |
| weight (g) | | | |
| Final | 9.48 +− 0.13^{c} | 10.38 +− 0.16^{b} | 10.71 +− 0.11^{a} |
| weight (g) | | | |
| Food | 2.49 +− 0.15^{c} | 2.06 +− 0.22^{b} | 1.54 +− 0.07^{a} |
| Conversion | | | |
| Factor (FCR) | | | |
| Survival (%) | 96.67 +− 3.0^{a} | 96.67 +− 3.87^{a} | 100^{a} |

The results of charts 5 and 7 show that the NRRL B-50213 strain and the formulas used are effective to improve growth, weight gain and percentage of survival, since they are an innovation and have an inventive activity since, to this date, there are no strains and formulas reported with this characteristics, that generate these benefits in carnivorous fish and shrimp.

### EXAMPLE 3

Increase in Energy Sources and Immunology Parameters in Fish and Shrimp

Fish and shrimp farms using formulas containing high concentrations of soy flour and/or starch and the NRRL B-50213 strain, improved their physiological and immunological parameters, when compared to those that were treated with commercial food or basal diet without the NRRL B-50213 strain. Chart 8 shows the levels of glucose, lactate, cholesterol and hemocites for shrimp and; glucose, red cells and hemoglobin for sea bass. In this chart it may be seen that all the parameters observed, were increased with the diets that containing high concentrations of vegetal flours and NRRL B-50213 strain, which was very positive for growth, increase of weight and survival (chart 5 and 7).

CHART 8
Increase of energy sources and immunology parameters for white shrimp and white sea bass.

| Shrimp | | | Sea Bass | | |
|---|---|---|---|---|---|
| | | Commercial | Basal Diet + | | |
| Parameters | Basal Diet | Diet | B-50213 | DC Diet | DC22 Diet |
| Glucose | 0.470 +− 0.02^{b} | 0.452 +− 0.03^{b} | 0.675 +− 0.02^{a} | 80 +− 37^{b} | 168 +− 36^{a} |
| Lactate | 0.261 +− 0.02^{b} | 0.249 +− 0.03^{b} | 0.385 +− 0.03^{a} | | |
| Cholesterol | 0.134 +− 0.07^{b} | 0.163 +− 0.03^{b} | 0.323 +− 0.08^{a} | | |
| Hemocites | 9.4 × 10⁶ +− 0.15^{b} | 9.63 × 10⁶ −+ 0.12^{b} | 2.02 × 10⁷ −+ 0.08^{a} | | |
| Red cells | | | | 1.5 +− 0.3^{b} | 2.0 +− 0.3^{a} |
| Cel ml⁻¹ | | | | | |
| Hb G dL⁻¹ | | | | 12.5 +− 1.8^{b} | 14.3 +− 2.3^{a} |

In light of the above, the NRRL B-50213 strain and our formulas are an innovation, since beyond of improving growth and weight gain, they increase the energy sources in the animal causing proteins to be used for growth and not as sources of energy.

In addition, the increase of the immunology parameters improved their resistance to illnesses produced by pathogen microorganisms and increased their percentage of survival (Charts 5, 7 and 8).

### EXAMPLE 4

Inhibition of Pathogen Bacteria on Fish and Shrimp, Using the NRRL B-50213 Strain

In solid means, opportunist pathogens were grown that are common to fish and shrimp (aeromones sp and Vibiro sp), against which the aliquot of the strain NRRL B-50213 were proven. Chart 9, shows that the NRRL B-50213 strain, subject matter of this patent was able to inhibit the growth of both pathogens during a 36 hour testing at 20° C. and 30° C., respectively.

CHART 9
Inhibition of pathogen bacteria for fish and crustaceans
in the presence of the NRRL B-50213 strain.

| Strain | Aeromona sp | Vibrio sp |
|---|---|---|
| B subtilis NRRL B- | +++ | +++ |
| 50213 | | |
| B subtilis W168 | + | + |

Area of inhibition in centimeters (2.0-1.0) +++ (1.0-0.5) ++ (0.5 -0.1) +

The results obtained indicate that beyond the positive effect of stimulation of the immunological parameters in fish and shrimps as induced by the formulas which are the subject matter of this patent (chart 8), the NRRL B-50213 strain by itself, can prevent illnesses caused by pathogen bacteria and as such, keep the farm healthy.

### EXAMPLE 5

Weight Gain and Survival of Pigs Using High Contents of SBM, Starch (Corn and/or Wheat Four) and the NRRL B-50213 Strain

In a commercial farm 300 pigs were evaluated at weaning, that is since they were 7 days old until they were 15 weeks old, the animals were fed with a formula that contained high concentrations of soy, corn and/or wheat and the NRRL B-50213 strain.

Historically this farm does not reach the ideal average growth rates that are between 25 and 55 kg up to the 10 to 15 weeks respectively. In addition, this farm and the whole state of Sonora, have a mortality rate of 17 to 20% at 15 weeks of age.

CHART 10
Weight gain of farmed pigs using high contents of SBM, starch
(corn and/or wheat flour) and NRRL B-50213 strain.

| | | |
|---|---|---|
| | Feeding without the | Feeding with the |
| Treatment | NRRL B-50213 strain | NRRL B-50213 strain |

Weaning Phase at 10 weeks
Ideal weight expected 25 kg

| | | |
|---|---|---|
| Initial weight (kg) | 7 | 7 |
| Final weight (kg) | 22 | 28 |

Phase at 10 to 15 weeks
Ideal weight expected 55 kg

| | | |
|---|---|---|
| Initial weight (kg) | 22 | 28 |
| Final weight (kg) | 45 | 56 |

The formula used to feed the control animals and the test animals was the same, except that the NRRL B-50213 strain was added to test animals. The results obtained from the trial and shown by chart 10, clearly show that using high concentration of vegetal flour as source of protein and energy and the NRRL B-50213 strain, can increase weight gain even more so than the average expected. In this trial, weight gain on the test animals was 11 kg above the control animals, which had no NRRL B-50213 strain.

CHART 11
Death rate decrease for farmed pigs using high contents of SBM,
starch (corn and/or wheat flour) and the NRRL B-50213 strain.

| | | |
|---|---|---|
| | Feeding without the | Feeding with |
| Treatment | NRRL B-50213 strain | NRRL B-50213 strain |

Weaning Phase at 10 weeks
Average Death rate 10%

| | | |
|---|---|---|
| % Initial Death rate | 0% | 0% |
| % Final Death rate | 10% | 5% |

Phase at 10 to 15 weeks
Average death rate 7%

| | | |
|---|---|---|
| % Initial Death rate | 10% | 5% |
| % Final Death rate | 7% | 2% |
| Total Death rate | 17% | 7% |

The formula used to feed the control animals and the test animals was the same, except that the NRRL B-50213 strain was added to test animals. The results obtained from the trial and shown by chart 11, clearly show that using a high concentration of vegetal flour as source of protein and energy and the NRRL B-50213 strain, up to a 10% death rate decrease can be prevented with respect to the control animals. In this trial, had a 7% death rate only compared to the control animals that had a 17% during the same period of study.

### REFERENCES

Amaya, E. A., Daves, D. A., Rouse, D. B. 1997a. Replacement of fish meal in practical diets for the Pacific white shrimp (Litopenaeus vannamei) reared under pond conditions. Aquaculture 262, 393-401.
Arellano-Carbajal, F y Olmos-Soto, J. 2002. Thermostable alfa-1-4 and alfa-1-6 glucosidase enzymes from Bacillus sp isolated from a marine environment. Journal of microbiology & Biotechnology 18: 791-795.
Arockia, A. J., Haniffa, M. A., Seetharaman, S. y Appelbaum, S. 2007. Utilization of various dietary carbohydrate levels by the freshwater catfish Mystus montanus (Jerdon). Turkish Journal of fisheries and aquatic sciences 8: 31-35.
Bautista-Teruel, M. N., Eusebio, P., Welsh, T., 2003. Utilization of feed pea, Pisum sativum, meal as a protein source in practical diets for juvenile tiger shrimp, Penaeus monodon. Aquaculture 225:121-131.
Cordel, C. T., 2004. Soy protein allergy: Incidence and relative severity. Am Soc Nutri Scis. 134:1213-1239.
Cuzon, G., Rosas, C., Gaxiola, G., Taboada, G. y Van Wormhoudt, A. 2000. Utilization of carbohydrates by shrimp. Memories of the fifth Symposium of underwater Nutrition.
Dunsford B R., Knabe, D A, Haensly W E. 1989. Effect of dietary soybean meal on the microscopic anatomy of the small intestine in the early-weaned pig. J Anim Sci 67:1855-1864.
Dersjant-Li, Y., 2002. The use of soy protein in aquafeeds. In: Cruz-Suarez, L. E., Ricque-Marie, D., Tapia-Salazar, M., Gaxiola-Cortes, M. G., Simoes, N (Eds) Avances en Nutricion Acuicola VI. Memories of the VI Symposium on underwater Nutrition. Sep. 3 to 6, 2002. Cancún, Quintana Roo, Mexico, pp. 541-558.
Enes, P., Panserat, S., Kaushik, S. C and Oliva-Teles, A. 2008. Growth performance and metabolic utilization of diets with native and waxy maize starch by gilthead sea bream (Sparus aurata) juveniles. Aquaculture 274:101-108.
FAO, 2006. The state of world fisheries and aquaculture, FAO Fisheries Department, Rome.
Forster, I P., Dominy, W., Tacon, A G J. 2002. The use of concentrates and other soy products in shrimp feeds. In: Cruz-Suarez, L. E., Ricque-Marie, D., Tapia-Salazar, M., Gaxiola-Cortes, M. G., Samos, N (Eds) Advances in Underwater Nutrition VI. Memories of the VI international symposium on underwater nutrition. Sep. 3 through 6, 2002. Cancun, Quintana Roo, Mexico.
Garcia, T. A. and Olmos, S. J. (2007) Quantification of fluorescent in-situ hybridization of bacteria associated with Litopenaeus vannamei larvae in Mexican shrimp hatchery. Aquaculture 262:211-218
German, D., Horn, M. y Gawlicka, A. 2004. Digestive enzyme activities in herbivorous and Carnivorous prickleback fish (Teleostei: Stichaeidae): Ontogenetic, Dietary and Phylogenetic effects. Physiological and Biochemical Zoology 77(5): 789-804.
Hardy, R. W., Barrows, F. T. 2002. Diet formulation and manufacture. In: Halver J. E. and Hardy R. W. Fish Nutrition, 3th edition. Academic Press, San Diego, USA 824 pp.
Heikkinen, J., Vielma, J., Kemilainen, O., Tiirola, M., Eskelinen, P., Kiuru, T., Mavia-Paldanius, D., Von Wright, A. 2006. Effects of soybean meal based diet on growth performance, gut histopathology and intestinal microbiota of juvenile rainbow trout (Oncorhynchus mykiss). Aquaculture 261:259-268.
Hemre, G. I., Shiau, S. Y., Deng, F. D., Storebakken, T y Hung, S. 2002. Utilization of hydrolyzed potato starch by juvenile Atlantic salmon when using a restricted feeding regime. Aquaculture Research 31: 207-212.
Krogdahl, A. Hemre, G. I. y Mommsen, T. P. 2005. Carbohydrates in fish nutrition: digestion and absorption in postlarval stages. Aquaculture nutrition 11: 103-122.
Kunst, F., Ogasawara, N., Moszer, I. and Albertini, A. M. (1997) The complete genome sequence of the gram-positive bacterium Bacillus subtilis. Nature 390:249-256
Kureshy, N., Davis, D. A., 2002. Protein requirements for maintenance and maximum weight gain for the pacific white shrimp. Litopenaeus vannamei, Aquaculture 204:125-143.
Le Chevalier, P., Van Wormhoudt, A., 1998. Alpha-glucosidase from the hepatopancreas of the shrimp, Penaeus vannamei (Crustaceas-Decapoda). J. Exp. Zool. 280:384-394.
Mahious, A. S., Gatesoupe, F. J., Hervi, M., Metailler, R. y Ollevier, F. 2006. Effect of dietary insulin and oligosaccharides as prebiotics for weaning turbot, Psetta maxima. Aquaculture 14(3): 219-229.
Nora, N T., Esther S D., Wisdom K A. 2006. The comparative ability of four isolates of Bacillus subtilis to ferment soybeans into dawadawa. Intl J Food Microbiol. 106:145-152.
Ochoa-Solano, L. y Olmos-Soto, J. 2006. The functional property of Bacillus for shrimp feed. Food microbiology 23: 519-525.
Olmos, S. J. and Contreras, F. R. (2003). Genetic system constructed to overproduce and secrete proinsulin in Bacillus subtilis. Appl Microbiol Biotechnol 62:369-373
Pascual, C., Arena, L., Cuzon, G., Gaxiola, G., Tabeada, g., Valenzuela, M., Rosas,c., 2004a. Effect of a size-based selection program on blood metabolites and immune response of Litopenaeus vannamei juveniles fed different dietary carbohydrate levels. Aquaculture 230:205-416.
Pascual, C., Zenteno, E., Cuzon, G., Sanchez, A., Gaxiola, G., Taboada, G., Suarez, J., Maldonado, T., Rosas, C., 2004b. Litopenaeus vannamei juveniles energetic balance and immunological response to dietary protein. Aquaculture 236:431-450.
Peres, H. y Oliva-Teles, A. 2002. Utilization of raw and gelatinized starch by European sea bass (Dicentrarchus labrax) juveniles. Aquaculture 205: 287-299.
Rosas, C., Cuzon, G., Gaxiola., Arena, I., Lemaire, P., Zoyez, C., Van Wormhoudt, A., 2000. Influence of dietary carbohydrate on the metabolism of juvenile Litopenaeus stylirostris. J Exp Mar Biol Ecol. 249:181-198.
Sambrook, K. T., Frisch, E. F. and Maniatis, T. (1989) Molecular cloning. A laboratory Manual. 2nd edn. Cold Spring Harbor Laboratory Press, Cold Spring Harbor, N.Y.
Shimeno, S., Hosokawa, H., Takeda, M., Kajiyama, H. y Kaisho, T. 1985. Effect of dietary and carbohydrate on growth, feed conversion and Body composition in young yellowtail. Bulletin of the Japanese Society of Scientific Fisheries 51 (1): 1893-1898.
Sonnenschein, A. L., Losick, R. and Hoch, J. A., (1993). Bacillus subtilis and Others Gram-Positive Bacteria: Biochemistry, Physiology and Molecular Genetics. American Society for Microbiology., Washington, D.C., pp. 987.
Suárez, M. D., Sanz, A., Bazoco, J. y Garcia-Gallego, M. 2002. Metabolic effects of changes in the dietary protein: carbohydrate ratio in eel (Anguila anguilla) and trout (Oncorhynchus mykiss). Aquaculture International 10: 143-156.
Tacon, A. G. J., Metian, M. 2008 Global overview on the use of fish meal and fish oil in industrially compounded aquafeeds. Trends and future prospects. Aquaculture, 285:146-158.
Vangen, B. y Hemre G. 2002. Dietary carbohydrate, iron and zinc interactions in Atlantic salmon (Salmo salar). Aquaculture 219: 597-611.
Wilson, R. P. 2002. Dicentrarchus labraxs, D. C., Lim, C (Eds). Nutrient Requirements and feeding of finfish for aquaculture. CABI Publishing. Pp 144-175.

Zhu Y P., Fan J F., Cheng Y Q y Li L T. 2008. Improvement of the antioxidant activity of Chinese traditional fermented okara (Meitauza) using Bacillus subtilis B2. Food Control. 19:654-661.

1. A strain of the Bacillus subtilis bacteria placed with the access number NRRL B-50213 and its mutants characterized as such because they also have the characteristics of the aforementioned strain. Also, the supernatant and the complete culture of the NRRL B-50213 strain.

2. The strain and/or its derivates (mutants, supernatant and complete culture) of claim 1, characterized since they show activity in the degradation of food.

3. The strain and/or its derivates of the claim 1, are characterized because the food contains percentages of starch of at least 12% eliminating the adverse effects of this ingredient.

4. The strain and/or its derivates, of claims 1, 2 and 3 are characterized because the food contains percentages of soy flour of at least in 20% eliminating the adverse effects of this ingredient.

5. The strain and/or its derivates, of claims 1, 2, 3 and 4 are characterized because the food has carbohydrates and/or protein and/or lipids of vegetal and/or animal origin.

6. The strain and/or its derivates, of claims 1, 2, 3, 4 and 5 are characterized because they increase the assimilation of food nutrients, since it promotes degradation by the production of enzymes.

7. The strain and/or its derivates, of claims 1, 2, 3, 4, 5 and 6 are characterized because of the increase of growth, weight gain and % of survival in animals.

8. The strain and/or its derivates, the previous claim are characterized because they have antimicrobial activity against pathogen microorganisms and improve the immune system of animals.

9. It is a food mix that is characterized because it contains the strain and/or its derivates of the claim 1.

10. The mix of claim 9 is characterized because the NRRL B-50213 strain and/or its derivates have an activity of food degradation.

11. The mix of claims 9 and 10, is characterized because it contains the complex polysaccharides of starch in a least 12%.

12. The mix of the claims 8, 9, 10 and 11, is characterized because it also contains vegetal flours such as soy flour in a least 20%.

13. The mix of claims 8, 9, 10, 11 and 12, is characterized because it contains composition of carbohydrates and/or proteins and/or lipids of vegetal and/or animal origin and possible combinations.

14. The mix of claim 9, is characterized by the antimicrobial activity of the NRRL B-50213 strain and/or its derivates against pathogen microorganisms and its stimulation of the immune system.

15. A food mix that is characterized by of the increase of nutrient assimilation of food, since it contains the strain and/or its derivates of claim 1.

16. A method to prepare a food mix, that is characterized by its content of the NRRL B-50213 strain and/or its derivates, complex polysaccharides as starch added at least 12% and/or vegetal flours such as soy flour added in at least 20% and/or protein and/or lipids of animal and/or vegetal origins and their possible combinations.

17. A method of increasing the degradation and assimilation of food characterized by the application of an effective amount of a food mix, that contains the NRRL B-50213 strain and/or its derivates; where the intake is made orally.

18. A method of increasing growth, weight gain and the percentage of survival of animals characterized by the application of an effective amount of a food mix, that contains the NRRL B-50213 strain and/or its derivates; where the intake is made orally.

19. A method to inhibit the growth of pathogen microorganisms and stimulation of the immune system characterized by the application of an effective amount of food mix, containing the NRRL B-50213 strain and/or its derivates; where intake occurs orally.

20. The use of the Bacillus subtilis strain under number NRRL B-50213 and/or its derivates in combination with other chemical and/or biological compounds, to prepare a food mix.
